(19) United States
(12) Patent Application Publication
(10) Pub. No.: US 2007/0027438 A1
(43) Pub. Date: Feb. 1, 2007

(54) System and method for compensating a corneal dissection

(76) Inventors: Frieder Loesel, Mannheim (DE); Tobias Kuhn, Heidelberg (DE)

Correspondence Address: NEIL K. NYDEGGER;NYDEGGER & ASSOCIATES, 348 Olive Street, San Diego, CA, 92103, US

(21) Appl. No.: 11/189,426

(22) Filed: Jul. 26, 2005

(51) Int. Cl. A61B 18/18

(52) U.S. Cl. 606/4

(57) ABSTRACT

A system and method for dissecting a transparent material utilizes pre-dissection diagnostic information about the transparent material. Specifically, in the system and method, a prototypic dissection path is planned to achieve a desired result. Then, the topology of the transparent material is defined and analyzed to calculate a predicted result of a dissection along the prototypic dissection path. After comparing the desired result and the predicted result, a refined dissection path is established in which any difference between the predicted result of a dissection along the refined dissection path and the desired result is minimized. As a result, dissection of the transparent material along the refined dissection path achieves the desired result.

### FIELD OF THE INVENTION

The present invention pertains generally to systems and methods used in corrective optical surgery. More particularly, the present invention pertains to systems and methods that dissect corneal tissue during a corrective optical operation. The present invention is particularly, but not exclusively, useful as a system and method in which the anatomical conditions of a patient's eye are used to predict the effects of pre-existing conditions in the corneal tissue and the effects of the dissection upon the corneal tissue to provide a dissection plan that compensates for such effects.

### BACKGROUND OF THE INVENTION

In the perfect eye, an incoming beam of light is focused through the cornea and through the crystalline lens in a way that causes all of the light from a point source to converge at the same spot on the retina of the eye. This convergence occurs because all of the optical path lengths, for all light in the beam, are equal to each other. Stated differently, in the perfect eye, the time for all light to transit through the eye will be the same regardless of the particular path that is taken by the light.

Not all eyes, however, are perfect. The consequences of this are that light path lengths through the eye become distorted and are not all equal to each other. Thus, light from a point source that transits through an imperfect eye will not necessarily be focused on the retina, or to the same spot on the retina.

Normally, as light enters and passes through an eye it is refracted at the anterior surface of the cornea, at the posterior surface of the cornea, and at the surfaces of the crystalline lens. After all of these refractions have occurred, the light finally reaches the retina. As indicated above, in the case of the perfect eye, all of these refractions result in no overall change in the optical path lengths of light in the incoming beam. Therefore, any deviations resulting in unequal changes in these optical path lengths are indicative of imperfections in the eye that may need to be corrected.

In general, vision difficulties in the human eye can be characterized by the changes and differences in optical path lengths that occur as light transits through the eye. These difficulties are not uncommon. Indeed, nearly one half of the world's population suffers from imperfect visual perception. For example, many people are nearsighted because the distance between the lens and retina is too long (myopia). As a result, the sharp image of an object is generated not on the retina, but in front of or before the retina. Therefore, for a myopic person a distant scene appears to be more or less blurred. On the other hand, hyperopia is a condition wherein the error of refraction causes rays of light entering the eye parallel to the optic axis to be brought to a focus behind the retina. This happens because the distance between the lens and retina is too short. This condition is commonly referred to as farsightedness. Unlike the myopic person, a hyperopic, or farsighted, person will see a near scene as being more or less blurred.

Another refractive malady is astigmatism. Astigmatism, however, is different than either myopia or hyperopia in that it results from an unequal curvature of the refractive surfaces of the eye. With astigmatism, a ray of light is not sharply focused on the retina but is spread over a more or less diffuse area.

Further, in addition to the more simple refractive errors mentioned above, the human eye can also suffer from higher order refractive errors (“aberrations”) such as coma, trefoil and spherical aberration. More specifically, coma is an aberration in a lens or lens system whereby an off-axis point object is imaged as a small pear-shaped blob. Coma can be described as a wavefront shape with twofold symmetry and is caused when the power of the zones of the lens varies with distance of the zone from the axis. Likewise, trefoil is described as a wavefront shape having threefold symmetry. Spherical aberration results from loss of definition of images that are formed by optical systems, such as an eye. Such aberrations arise from the geometry of a spherical surface. For these higher order aberrations (“HOAs”), an ideally flat ‘wavefront’ (i.e. a condition wherein all optical path lengths are equal) is distorted by a real-world optical system. In some cases, these distortions occur in a very complex way. In the trivial case, non-higher order distortions like nearsightedness and farsightedness would result in an uncomplicated bowl-like symmetrical distortion. With HOAs, however, the result is a complex non-symmetrical distortion of the originally flat wavefront. It is these non-symmetrical distortions which are unique for every optical system (e.g., a person's eye), and which lead to blurred optical imaging of viewed scenes.

While a typical approach for improving the vision of a patient has been to perform refractive surgery on the eye to eliminate distortions, typically, the surgery does not compensate for pre-existing HOAs. Further, the surgery itself can lead to an increase in HOAs, both immediately and during recovery. Indeed, it has been determined that conditions such as biomechanical stress distribution and hydration levels can induce changes in the optical characteristics of an eye as a mere consequence of corneal dissection. Specifically, dissection of the cornea can induce HOAs including vertical coma, horizontal coma, spherical aberration and 90/180° astigmatism. Further, because each eye has its own distinct physical characteristics, identical dissections performed in two different eyes lead to distinctly different results.

In light of the above, it is an object of the present invention to provide a system and method that defines the topology of the cornea, or other transparent material, in order to predict the result of a dissection of the corneal tissue. Another object of the present invention is to provide a system and method that incorporates the anatomical conditions in the cornea into surgical planning to compensate for pre-existing HOAs and for the effects of the dissection on the cornea. Another object of the present invention is to provide a system and method that incorporates pre-dissection wavefront data into the dimensional planning of the dissection. Still another object of the present invention is to provide a system and method for predicting and precompensating for changes in the corneal tissue induced by dissection which are effectively easy to use, relatively simple to operate and implement, and comparatively cost effective.

### SUMMARY OF THE INVENTION

In the present invention, a system is provided for dissecting a transparent material, such as in the cornea of an eye, via photoablation. More specifically, the system of the present invention dissects the transparent material while compensating for pre-existing topological conditions, as well as effects otherwise induced by topological conditions of the transparent material during dissection.

Structurally, the system of the present invention includes two distinct laser sources. One is for generating a diagnostic laser beam. The other is for generating an ablation laser beam that will be used to photoablate corneal tissue during creation of the flap. Along with the two laser sources, the system typically includes an active mirror and a detector. More specifically, the active mirror comprises a plurality of separate reflective elements for individually reflecting respective component beams of the diagnostic beam. Together, these elements of the active mirror are used, in concert, to direct the diagnostic laser beam to a focal spot on the retina of the eye. The detector is then used to receive the diagnostic beam after it has been reflected from the retina. The system further includes a comparator and compensator that are used with the detector during operation of the ablation laser beam, as discussed below.

In the operation of the present invention, diagnostic measurements are initially made. Specifically, the distorted wavefront of the patient's eye is measured. To do this, the diagnostic laser beam is passed through the patient's eye, reflected by the patient's retina and received by the detector. The reflected laser beam is properly considered to include a plurality of individual component beams. Collectively, these constituent component light beams define a wavefront for the larger inclusive light beam. For the present invention, the wavefront that is received by the detector, and that results from passing through the stroma of an uncorrected eye is considered to be a “distorted wavefront.” Thus, a distorted wavefront exhibits the actual real-time characteristics of the cornea.

In view of the distorted wavefront, a desired result from the corrective operation can be specified. Typically, the desired result will be characterized by a wavefront which is planar or substantially planar. After the desired result is specified, the volume of corneal tissue to be ablated to achieve the desired result is determined. In certain cases, the desired result is specified and the volume of tissue to be ablated is determined with the understanding that a prosthetic will be introduced into the cornea.

In addition to measuring the distorted wavefront of the patient's eye, wavefront analysis is performed to define the topology of the patient's cornea. As used herein, “topology” means all physical characteristics of the cornea, or other transparent material, and preferably includes stromal bed thickness, total corneal pachymetry, optical density, characteristics affecting biomechanical stresses in the cornea, and dimensions of the planned dissection.

Based on the distorted wavefront, a prototypic dissection path for dissection of the corneal tissue is identified. Specifically, the prototypic dissection path is identified through comparison of the distorted wavefront measured by the diagnostic laser beam and a desired wavefront. The prototypic dissection path typically bounds the previously determined volume of corneal tissue to be ablated. For the purposes of the present invention, the distorted wavefront is obtained as disclosed above, and the “desired wavefront” is planar or substantially planar. In any event, the desired wavefront is the objective of the optical correction operation. As envisioned for the present invention, during the identification of the prototypic dissection path, no consideration is given to the topology of the cornea.

Once the topology of the cornea has been defined and the prototypic dissection path has been identified, the two are then used together to calculate a predicted result of a dissection along the prototypic dissection path. The predicted result is then compared to the desired result. If the predicted result and the desired result differ, then the prototypic dissection path must be refined to compensate for the predicted effects of the cornea's topology and to establish a refined dissection path in which the effects of the cornea's topology are eliminated or minimized. In this manner, the present invention compensates for the predicted effects of the cornea's topology.

For the present invention, the refinement of the prototypic dissection path is essentially a two-step process. In the first step, the prototypic dissection path is refined in order to eliminate or minimize the inducement of HOAs that may result during the corrective operation. In the second step, the refined dissection path may then be even further refined to correct for pre-existing HOAs or other topological conditions. In this manner, the present invention compensates for both topological and anatomical effects, including those effects on the distribution of stress within the cornea and on the shape of the wound created during dissection.

In accordance with the present invention, the initial prototypic dissection path is essentially established as a path between two selected points in the cornea. This path may or may not be linear, and it is established as a succession of substantially contiguous locations where laser induced optical breakdown (LIOB) is accomplished. As indicated above, this initial prototypic dissection path is identified for the purpose of performing the required refractive surgery. As also indicated above, in order to account for HOAs, the prototypic dissection path needs to be refined. Depending on topological and anatomical considerations, refinement of the prototypic dissection path may require a two-step process as suggested above.

For the present invention, HOAs that may be induced when corneal tissue is cut during a corrective operation are minimized or eliminated by appropriately altering the course of the prototypic dissection path. In particular, by altering the course of the prototypic dissection path, the refined dissection path can be established to accommodate the redistribution of biomechanical stresses in the cornea that would otherwise result when the corneal tissue is cut. Without more, however, course alteration will not correct for the pre-existing HOAs.

If an evaluation of the cornea and the eye reveals pre-existing HOAs, the refined dissection path discussed above needs to be further refined. Specifically, using the initially refined dissection path as a base line, further refinement of the refined dissection path requires performing additional LIOB. In particular, the necessary additional LIOB is performed at lateral locations that are directed perpendicularly from selected points on the prototypic dissection path. As envisioned for the present invention, this additional LIOB is intended to remove tissue that will correct the non-induced (pre-existing) HOAs. Stated differently, if used, the second step in creating the refined dissection path results in altering the actual width of the prototypic dissection path to account for the non-induced (pre-existing) HOAs.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic layout showing the interrelationships of components in a system for customizing a corneal dissection in accordance with the present invention;

FIG. 2 is a functional representation of the wavefront analysis techniques used in the operation of the system of the present invention;

FIG. 3 is a functional flow chart illustrating the method for customizing a corneal dissection in accordance with the present invention;

FIG. 4A is a cross-sectional view of a cornea showing a prototypic and refined dissection path for a refractive surgery technique in accordance with the present invention;

FIG. 4B is a cross-sectional view of a cornea showing a prototypic and refined dissection path for another refractive surgery technique in accordance with the present invention; and

FIG. 4C is a cross-sectional view of a cornea showing a prototypic and refined dissection path for another refractive surgery technique in accordance with the present invention.

### DESCRIPTION OF THE PREFERRED EMBODIMENTS

Referring initially to FIG. 1, a system for dissecting a transparent material, e.g., corneal tissue, in accordance with the present invention is shown and is generally designated 10. In detail, the components of system 10 include a source 12, such as a femtosecond laser, for generating an ablation laser beam 14, and a source 16 for generating a diagnostic laser beam 18. Further, the system 10 includes an active, multi-facet mirror 20, a beam splitter 22 and a beam splitter 24. More particularly, the active mirror 20 is preferably of a type disclosed in U.S. Pat. No. 6,220,707, entitled “Method for Programming an Active Mirror to Mimic a Wavefront,” which is assigned to the same assignee as the present invention. As shown, the active mirror 20 and the beam splitters 22 and 24 direct the diagnostic laser beam 18 from the diagnostic laser source 16 toward an eye 26. Likewise, the beam splitters 22 and 24 are used to direct the ablation laser beam 14 from the ablation laser source 12 toward the eye 26.

FIG. 1 also shows that the system 10 of the present invention includes a detector 28, a comparator 30 and a compensator 32. In particular, the detector 28 is preferably of a type commonly known as a Hartmann-Shack sensor. The comparator 30 and compensator 32 are electronic components known in the pertinent art that will perform the requisite functions for the system 10.

In the present invention, the system 10 is used to make initial diagnostic evaluations of a patient's cornea 34, and in particular its stromal tissue 36. Specifically, the diagnostic laser beam 18 is focused (by optical components not shown) to a focal spot 38 on the retina 40 of the patient's eye 26. As shown in FIG. 1, the reflected diagnostic laser beam 18′ passes through the cornea 34, exits the eye 26, and is directed by the beam splitter 24 toward the detector 28. Using wavefront analysis, the system 10 analyzes the reflected diagnostic laser beam 18′ received by the detector 28 to measure the distorted wavefront 42 of the uncorrected eye 26.

When using wavefront analysis considerations, the reflected diagnostic beam 18′ is conceptually considered as including a plurality of individual and separate laser beam components. Together, these components are characterized as a distorted wavefront 42 that results from the uncorrected eye 26 as a consequence of light passing through the stromal tissue 36. FIG. 1 further shows an induced wavefront 44 and a desired wavefront 46. The induced wavefront 44 is generated by the detector 28 during photoablation of corneal tissue. As discussed below, the induced wavefront 44 results from the formation of bubbles during photoablation in the stroma. Typically, the desired wavefront 46 is either a plane wavefront, or a wavefront that is substantially similar to a plane wavefront.

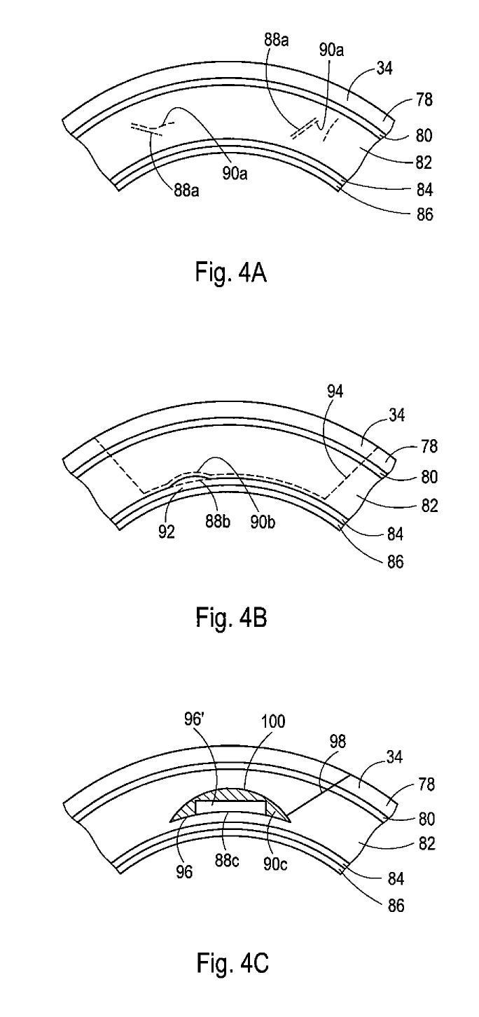

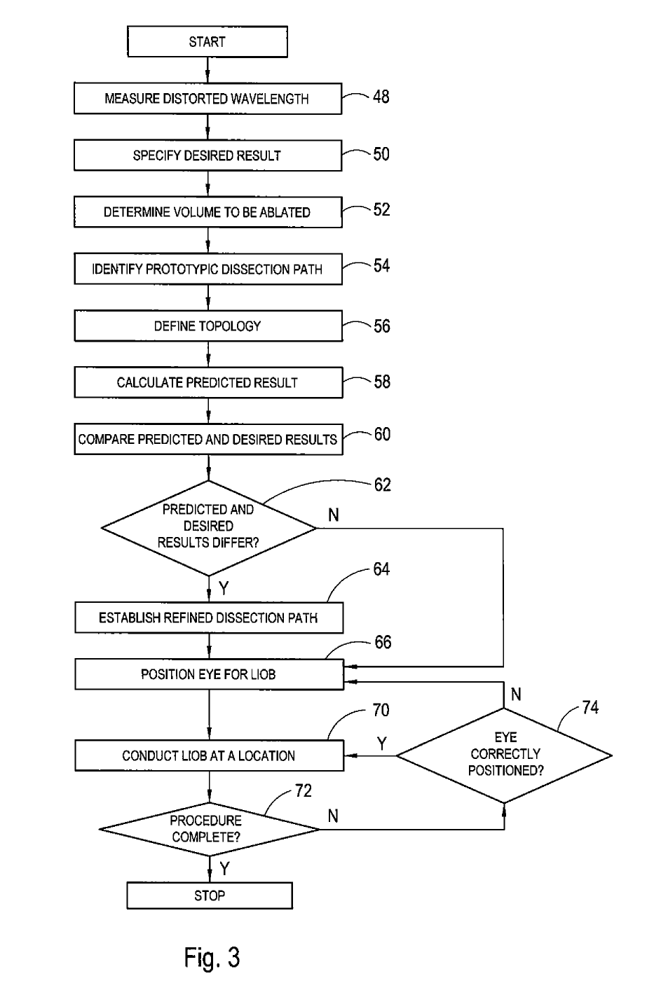

Referring now to FIG. 3, the operation of the system 10 is set forth. As discussed above and shown in action block 48, the system 10 first measures the distorted wavefront 42 of the eye 26 via wavefront technology. Next, as shown at action block 50, the system 10 specifies a desired result of the vision correction operation. Typically, the desired result is characterized by a desired wavefront 46 that is planar or substantially planar. As discussed in further detail below, specification of the desired result may take into consideration a specific technique to be used during the corrective operation.

At action block 52 in FIG. 3, the volume of corneal tissue to be photoablated is determined in accordance with the desired result. As discussed further below, this determination is dependent on the technique employed during the corrective operation. For example, the planned surgery may be an astigmatic keratotomy, a keratoplasty, or it may involve the removal of a lenticular volume of cornea. As further shown in FIG. 3, after the volume of corneal tissue to be ablated is determined, a prototypic dissection path is identified (action block 54). Such identification is based on a comparison between the distorted wavefront 42 and the desired wavefront 46 as depicted in FIG. 1. The determination in action block 52 and the identification in action block 54 are made without consideration of the topology of the cornea 34. Specifically, these steps are performed with the goal of correcting lower-order aberrations such as myopia, hyperopia, and/or astigmatisms.

Independent of the determination and specification steps of action blocks 52 and 54, the topology of the cornea 34 is defined in action block 56. In this step, wavefront analysis of the reflected diagnostic beam 18′ (shown in FIG. 1) is further utilized to define the topology of the cornea 34. As stated above, the “topology” of the cornea 34 refers to the cornea's physical properties, including stromal bed thickness, total corneal pachymetry, optical density, the biomechanical stress distribution in the cornea, as well as the dimensions of the planned dissection. Such properties are ascertained from the reflected diagnostic beam 18′. While wavefront technology is used to define the topology of the cornea 34 in the presently described embodiment, other techniques, such as ellipsometry, second harmonic generation (SHG) microscopy, confocal microscopy, corneal topography, optical coherence tomography (OCT), or ultrasonic pachymetry, may be used. In any case, after the topology of the cornea 34 is defined, it is used during corrective operation planning as discussed below.

Based on the topology defined in action block 56 and the prototypic dissection path identified in action block 54, the system 10 calculates a predicted result of a dissection along the prototypic dissection path as shown at action block 58. Essentially, the topological conditions in the eye 26 are analyzed so that their effects on the result of a dissection along the prototypic dissection path are known. The desired result is then modified with the predicted effects of the topological conditions to calculate the predicted result. Once the predicted result is calculated, it is compared to the desired result, as shown at action block 60.

As indicated by inquiry block 62, if the predicted result does not differ from the desired result, i.e., if no topological effects are predicted, then no further pre-operation steps are needed and the correction operation may commence. However, if the predicted result does differ from the desired result, then the prototypic dissection path must be refined to compensate for the predicted effects of the cornea's topology. As a result, a refined dissection path is established in which the effects of the cornea's topology are eliminated or minimized as shown at action block 64.

Establishing the refined dissection path involves a two-step process. In the first step, the prototypic dissection path is refined in order to eliminate or minimize the inducement of HOAs that may result during the corrective operation. In particular, the course of the prototypic dissection path is altered to establish the refined dissection path to accommodate the redistribution of biomechanical stresses in the cornea that would otherwise result when the corneal tissue is cut. In the second step of the process, the refined dissection path is even further refined to correct for pre-existing HOAs or other topological conditions. Specifically, using the initially refined dissection path as a base line, further refinement of the refined dissection path requires the identification of additional corneal tissue to be photoablated. In particular, the necessary additional LIOB is performed at lateral locations that are directed perpendicularly from selected points on the refined dissection path. As envisioned for the present invention, this additional LIOB is intended to remove tissue to correct the non-induced (pre-existing) HOAs and results in altering the actual width of the dissection path.

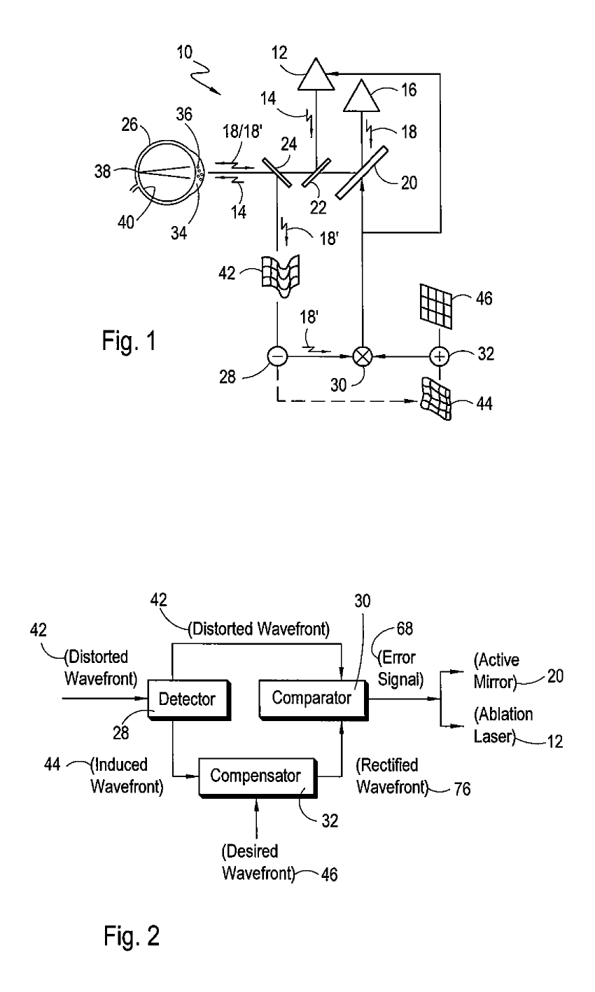

After the refined dissection path is established, refractive surgery may be performed. To prepare for surgery, the patient is positioned such that the system 10 and the eye 26 are generally in the same relative position as when the initial diagnosis was made (action block 66). In order to ensure proper positioning, a real-time, closed-loop, adaptive-optical control system as shown in FIG. 2 may be used. Specifically, as discussed above, a diagnostic laser beam 18 is focused on the patient's retina 40. The diagnostic laser beam 18′ reflected therefrom is directed to the detector 28 as a distorted wavefront 42. This distorted wavefront 42 is compared to the initially diagnosed distorted wavefront (not shown, but known by comparator 30) to generate an error signal 68. In response to the error signal 68, the relative position of the patient's eye 26 and the system 10 is modified. Then, the system 10 passes another diagnostic laser beam 18 through the eye 26 to measure a “new” distorted wavefront 42. This process is continued until it is concluded that the eye 26 is in the same relative position with respect to the system 10 as during the diagnosis.

Referring to FIG. 3, it is seen that, after the eye 26 is properly positioned, laser induced optical breakdown (LIOB), or photoablation, is conducted at a location along the refined dissection path (action block 70). Specifically, the ablation laser beam 14 is directed to a focal point along the refined dissection path to cause photoablation. While photoablation is preferably used, the present invention contemplates that any type of dissection may be performed.

As shown in inquiry block 72, if the procedure is complete after photoablation of the corneal tissue at the targeted location, i.e., if dissection is complete, then the corrective operation is stopped. If, however, the procedure is not complete, then further photoablation is required. As shown in inquiry block 74, before further photoablation occurs, it is determined whether the eye 26 is still properly positioned. If it is not, the eye 26 is re-positioned at action block 66. If the eye 26 is correctly positioned, the system 10 directs the ablation laser beam 14 to a different location along the refined dissection path and conducts LIOB at the new location. In order to ensure proper photoablation along the refined dissection path, the system 10 controls the location of the focal point of the ablation laser beam 14 in response to the detector's receipt of the distorted wavefronts 42 from the reflected diagnostic laser beam 18′. In other words, the continuously updated distorted wavefronts 42 show what locations in the cornea 34 have been fully photoablated. As a result, the system 10 moves the focal point of the ablation laser beam 14 to locations along the refined dissection path that still require photoablation. This process is repeated until the dissection of the corneal tissue is completed. While the system 10 is illustrated as using wavefront technology, it is contemplated herein that other measurement techniques such as ellipsometry, second harmonic generation microscopy, confocal microscopy, or other techniques can be used to provide monitoring of the dissection.

The present invention may include an optional operational loop that is of particular importance when bubbles formed in the stromal tissue 36 may affect HOAs. It is to be appreciated and understood that during an intrastromal photoablation procedure, gas bubbles form as a consequence of photoablation of the stromal tissue 36. When bubbles formed in the stromal tissue 36 do not collapse, they cause aberrations that affect the distorted wavefront 42 received by the detector 28. Based on the topology of the cornea 34, the collapse of bubbles formed in the stromal tissue 36 may be predicted. However, if a bubble behaves differently than as predicted, HOAs may be affected. In cases where bubbles do not behave as predicted, the refined dissection path is re-established at action block 64 in order to take into consideration such behavior. If the bubbles behave as predicted, the refined dissection path is not re-established.

Referring now to FIG. 2, it will be appreciated that in the operation of the system 10 the detector 28 first receives the distorted wavefront 42. Using the refined dissection path and the predicted bubble behavior, the detector 28 generates an induced wavefront 44. As used herein, an “induced wavefront” results from the formation of bubbles in the stroma, and includes the distorted wavefront 42. The compensator 32 then alters the predetermined, desired wavefront 46 with this induced wavefront 44. This alteration creates a rectified wavefront 76. As used herein, the “rectified wavefront” results from incorporating an induced wavefront with a desired wavefront. The rectified wavefront 76 is then compared with the distorted wavefront 42 to generate an error signal 68. In turn, this error signal 68 is used to manipulate the active mirror 20 for control of the diagnostic laser beam 18. Importantly, the error signal 68 is also used to activate the ablation laser source 12 and, specifically, the error signal 68 causes the ablation laser source 12 to cease its operation when the error signal 68 is a null.

Referring now to FIGS. 4A-4C, the techniques used during dissection are explained more fully. These figures depict a cross-sectional view of a cornea 34 to show its internal structure. As shown, the cornea 34 includes an epithelium 78, Bowman's membrane 80, stroma 82, Descemet's membrane 84, and endothelium 86.

Referring to FIG. 4A, a prototypic dissection path 88a is shown. Such a path 88a is used in astigmatic keratotomy to modify the properties of the cornea 34 in its mid-periphery. Specifically, volumes of cornea 34 are photoablated to change the biomechanical stress distribution within the eye 26. As indicated above, the volume of tissue to be ablated is determined and a prototypic dissection path 88a is identified without reference to the topological conditions of the cornea 34. After defining the topology of the cornea 34 and analyzing its effects, the prototypic dissection path 88a is refined to establish a refined dissection path 90a along which photoablation will occur. As shown in FIG. 4A, the refined dissection path 90a differs slightly from the prototypic dissection path 88a in order to compensate for the topological factors.

Referring now to FIG. 4B, a prototypic dissection path 88b is shown for a non-perforating, deep lamellar keratoplasty. In this procedure, a type of deep flap is cut in the cornea 34 and then removed and replaced by a prosthetic (not shown). For the purposes of the present invention, the prosthetic may comprise artificial or biological material such as a donor cornea. In deep lamellar keratoplasty, the cut should follow the posterior boundary of the stroma 82 without damaging the posterior layers of the cornea 34, i.e., Descemet's membrane 84 and the endothelium 86. As shown, the prototypic dissection path 88b intersects an irregular portion 92 of Descemet's membrane 84. After the refining step described in reference to FIG. 3, a refined dissection path 90b is established to closely follow the boundary of Descemet's membrane 84 without piercing it. In the deep lamellar keratoplasty procedure, the prosthetic should have a shape that mates with the corneal wound 94. In order to properly prepare the prosthetic, the system 10 is used to predict the shape of the corneal wound 94 and to cut the prosthetic to fit the corneal wound 94 precisely.

Referring to FIG. 4C, a cornea 34 undergoing another refractive surgery technique is shown. For this technique, a lenticular volume 96 is cut into the stroma 82 and removed from the cornea 34 through a slit 98 in the periphery of the cornea 34. Because the outer parts of the lenticular volume 96 are only a few microns or even fractions of a micron thick, removal of the lenticular volume 96 is typically difficult. In order to overcome this, the prototypic dissection path 88c is refined to establish the refined dissection path 90c in which a larger portion 100 of the lenticular volume 96 is photoablated. This process leaves a smaller, but more easily grasped, lenticular volume 96′ to be removed through the slit 98. When the refined dissection path 90c is established, effects caused by the topology of the cornea 34 are considered as in the techniques discussed above. In this manner, the process involving the removal of the lenticular volume 96′ compensates for topological conditions in the cornea 34.

While the particular system and method for Compensating a Corneal Dissection as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims. It will be appreciated that the systems and methods of the present invention can be applied to any transparent material.

What is claimed is:

1. A system for dissecting a transparent material which comprises:
means for specifying a desired result from a dissection;
means for determining a volume of transparent material to be altered during the dissection;
means for identifying a prototypic dissection path for the dissection;
means for defining a topology for the transparent material;
means for calculating a predicted result of the dissection based on the topology;
means for refining the prototypic dissection path to establish a refined dissection path, wherein the refined dissection path minimizes a difference between the predicted result and the desired result; and
means for dissecting the transparent material along the refined dissection path.

2. A system as recited in claim 1 wherein the refined dissection path minimizes HOAs induced during dissection of the transparent material and corrects for pre-existing HOAs in the transparent material.

3. A system as recited in claim 1 wherein the refined dissection path bounds the volume of transparent material to be altered and the system further comprises:
means for removing the volume of transparent material to create a recess in the transparent material; and
means for inserting a prosthetic into the recess.

4. A system as recited in claim 1 wherein the dissecting means creates incisions in the transparent material by laser induced optical breakdown.

5. A system as recited in claim 1 wherein the transparent material is corneal tissue and the topology is based on predictors including stromal bed thickness, dimensions of the prototypic dissection path, and total corneal pachymetry.

6. A system as recited in claim 5 wherein the predictors are used to define a biomechanical stress distribution and hydration levels in the corneal tissue.

7. A system for dissecting a transparent material which comprises:
means for determining a volume of transparent material to be altered during a dissection;
means for identifying a prototypic dissection path for the dissection;
means for defining a topology for the transparent material;
means for refining the prototypic dissection path to establish a refined dissection path, wherein the refined dissection path compensates for optical aberrations otherwise induced by the topology during the dissection; and
means for dissecting the transparent material along the refined dissection path.

8. A system as recited in claim 7 wherein the refined dissection path bounds the volume of transparent material to be altered and the system further comprises:
means for removing the volume of transparent material to create a recess in the transparent material; and
means for inserting a prosthetic into the recess.

9. A system as recited in claim 8 wherein an interface is formed between the prosthetic and the transparent material, and the system further comprises means for reforming the transparent material at the interface for compliance with the determining means.

10. A system as recited in claim 7 wherein the system further comprises means for altering the enclosed volume by laser induced optical breakdown.

11. A system as recited in claim 7 wherein the transparent material is corneal tissue and the topology is based on predictors including stromal bed thickness, dimensions of the prototypic dissection path, and total corneal pachymetry.

12. A system as recited in claim 11 wherein the predictors are used to define a biomechanical stress distribution and hydration levels in the corneal tissue.

13. A method for dissecting a transparent material which comprises the steps of:
specifying a desired result from a dissection;
determining a volume of transparent material to be altered during the dissection;
identifying a prototypic dissection path for the dissection;
defining a topology for the transparent material;
calculating a predicted result of the dissection based on the topology;
refining the prototypic dissection path to establish a refined dissection path, wherein the refined dissection path minimizes any difference between the predicted result and the desired result; and
dissecting the transparent material along the refined dissection path.

14. A method as recited in claim 13 wherein the refined dissection path bounds the volume of transparent material to be altered and the method further comprises the steps of:
removing the volume of transparent material to create a recess in the transparent material; and
inserting a prosthetic into the recess.

15. A method as recited in claim 14 wherein an interface is formed between the prosthetic and the transparent material, and the method further comprises the step of reforming the transparent material at the interface for compliance with the determining step.

16. A method as recited in claim 13 wherein the dissecting step encloses the volume of transparent material, with the method further comprising the step of altering the enclosed volume by laser induced optical breakdown.

17. A method as recited in claim 13 wherein the transparent material is corneal tissue and the topology is based on predictors including stromal bed thickness, dimensions of the prototypic dissection path, and total corneal pachymetry.

18. A method as recited in claim 17 wherein the predictors are used to define a biomechanical stress distribution and hydration levels in the corneal tissue.

19. A method as recited in claim 13 wherein the desired result is achieved through a reduction of higher order aberrations in the transparent material.

20. A method as recited in claim 13 wherein the desired result includes improved refractive performance by the transparent material.
